(19) United States
(12) Patent Application Publication
(10) Pub. No.: US 2026/0048095 A1
(43) Pub. Date: Feb. 19, 2026

(54) Treatment of Neuroinflammation

(71) Applicant: Jacek Kwiecien, Hamilton (CA)

(72) Inventor: Jacek Kwiecien, Hamilton (CA)

(21) Appl. No.: 18/805,174

(22) Filed: Aug. 14, 2024

(51) Int. Cl. A61K 36/899; A61K 31/121; A61K 38/16; A61P 25/00; A61P 29/00

(52) U.S. Cl. CPC A61K 36/899; A61K 31/121; A61K 38/168; A61P 25/00; A61P 29/00

(57) ABSTRACT

A method of treating or reducing neuroinflammation in a mammal is provided. The method comprises administration to the mammal of non-immunogenic protein having anti-inflammatory activity, wherein the protein comprises at least about 1 g/100 g protein of sulfur-containing amino acids selected from cysteine, methionine and a combination thereof.

### BACKGROUND OF THE INVENTION

Treatments for neurotrauma, stroke, neurodegenerative diseases, and immune mediated diseases of the central nervous system (CNS) are lacking or are generally not effective. This unfortunate status has not been helped by the poor understanding of the pathogenesis of these diseases with futile therapeutic strategies aimed at wrong pathologic targets. Neurotrauma in spinal cord injury (SCI) and in traumatic brain injury (TBI) that results in a locally massive necrosis and hemorrhage in the white matter, initiates a severe and extraordinarily protracted inflammation fueled by potently immunogenic damaged myelin. Although stroke is not initiated by a traumatic event but rather by an occlusive vascular accident in the brain, the resulting ischemia, local massive necrosis and sometimes hemorrhages involving the white matter, initiate the same inflammatory response. The pathogenesis of neurotrauma and stroke has recently been elucidated in a systematic study on the rat model of the SCI where macrophage-rich infiltration is directed at removing myelin-rich necrotic debris and red blood cells and also results in elevation of pro-inflammatory cytokines including IL-1B, IL-6 and IFN-gamma, and chemokines, and in damage to the spinal cord around the initial lesion. Since the human brain is rich in white matter content, and TBI and stroke often involve this myelin-rich tissue, the myelin-rich spinal cord is an appropriate model of the white matter injury in TBI and stroke. Rodent models of TBI and stroke involve primarily gray matter injury since the white matter content in the brain of mice and rats is low. A gray matter injury initiates inflammation that is much less severe and is self-limiting. Severe inflammation initiated by neurotrauma (SCI and TBI) and stroke involving the white matter, is associated with damage to small and capillary blood vessels in the CNS around the inflammatory lesion leading to leaking of excess fluid that can overwhelm water management by astrocytic systems leading to cerebral edema or spinal cord edema. It is considered that neuroinflammation initiated by TBI and SCI can be more damaging to neurological outcome than neurotrauma itself. Neurotrauma-initiated inflammation has been recognized in the animal models of SCI and in TBI and in human neurotrauma, with macrophages starting to infiltrate the site of injury by day 3. Inflammatory disease has not been studied systematically along its entire course until recently. Also, an anti-inflammatory and anti-edema tissue reaction in the spinal cord that appears to have a beneficial effect on neurologic function and to inhibit and ultimately eliminate macrophages from the site of trauma has not been previously addressed. The progressively severe astrocytic response to neurotrauma and stroke or astrogliosis appears to be the cellular reaction associated with anti-inflammatory and anti-edema effect in the SCI and the molecular mechanisms playing a role in these beneficial functions currently are unknown.

A cerebral vascular occlusion can cause ischemia and result in stroke. The resulting focal necrosis involving the white matter initiates severe, destructive, macrophage-rich inflammation not unlike that caused by SCI and TBI with rodent models of stroke used to study and to treat it. Long term outcome of cerebral neuropathology resulting from stroke can result in the development of Alzheimer's disease (AD), frontal temporal dementia (FTD), or Parkinson's disease.

Neuroinflammation involving activated, pro-inflammatory microglia and macrophages has been associated with neurodegeneration in progression of AD and in mouse models of AD. Despite failures of therapeutic strategies directed at removal of amyloid plaques and tau-rich neurofibrillary tangles (NF), more recent experimental anti-inflammatory treatments have been shown to inhibit the progression of cognitive decline and neuropathology in models of AD and to reduce markers of neuroinflammation and of cognitive decline in clinical trials. Frontal temporal dementia (FTD), a disease leading to cognitive decline and loss of cortical neurons but without deposition of amyloid and NF, therefore of distinct pathogenesis from AD, has also been associated with neuroinflammation involving activated microglia and pro-inflammatory macrophages. Therefore, anti-inflammatory treatments of this disease would be considered as potentially beneficial.

The pathogenesis of Parkinson's disease (PD) involves loss of dopaminergic neurons in the substantia nigra in the midbrain and related atrophy of striatal neurons. Neurodegeneration in PD has been associated with neuroinflammation involving activated microglia and pro-inflammatory macrophages with neuroinflammation resulting from a preceding TBI or stroke considered a highly contributing factor. Therefore, anti-inflammatory treatments of this disease would be considered as beneficial.

The pathogenesis of amyotrophic lateral sclerosis (ALS) involves a rapid degeneration of spinal motor neurons and specific neurons in the brain cortex and subcortical nuclei integrated in motor function. The neuroinflammation with activation of microglia and infiltration by pro-inflammatory macrophages in the spinal and cerebral areas has been documented in human patients and in SOD-1 mouse mutants. Anti-inflammatory treatments of this disease would be considered as beneficial.

Immune-mediated myeloencephalitides, inflammatory diseases of the spinal cord and the brain include multiple sclerosis (MS), and neuromyelitis optica (NMO). Specific antigens against which the immune reaction in MS is mounted are still unknown. In NMO, auto-antibodies directed against the aquaporin-4, a water channel in astrocytic cell membrane initiate a severe, rapidly progressing NMO. Although some forms of early MS involve perivascular inflammation that recedes after a period of time, chronic MS tends to result in a more severe, parenchymal inflammation that involves activated microglia and pro-inflammatory macrophages active in demyelinating plaques. Immunosuppressive treatments of MS have been used with variable, often inadequate outcomes. Anti-inflammatory treatments of this disease would be beneficial.

A number of viral diseases of man can result in severe neuroinflammation with resulting damage to the blood brain barrier and neuronal loss. Although, the CNS is particularly well protected against infectious agents gaining entry via the blood stream, bacterial, fungal, protozoal and larval parasitic infections can cause devastating neuroinflammation with destruction of neuropil and related neurologic deficits or death. Anti-inflammatory treatments would provide neuroprotection in these diseases.

While all neuroinflammatory diseases discussed above are or are expected to be associated with vascular damage and cerebrospinal edema, this obvious relation has not been addressed until recently. Anti-inflammatory agents therefore, are expected to inhibit vascular damage, lead to reduced fluid leakage thus limiting cerebral and spinal edema. Currently, there are no satisfactorily effective treatments of all diseases discussed above while the abundance of supportive scientific evidence on inflammation is at the core of pathogenesis of neurotrauma, stroke, neurodegenerative and infectious diseases and indicates that effective anti-inflammatory treatments would address this shortfall.

The inhibition and elimination of the severe inflammation initiated by neurotrauma (SCI and TBI) and stroke is the first and necessary step in treatment of these diseases. By inhibiting inflammation, the inflammatory damage to cerebral and spinal cord blood vessels would be reduced and/or eliminated, and the elimination of edema fluid accelerated. Based on the understanding of the pathogenesis of neurotrauma (SCI, TBI) and stroke, anti-inflammatory agents have been infused subdurally in the vicinity of the SCI lesion in the rat model and reduction of macrophage numbers in the lesion associated with improvement of neurologic deficits was observed. Although inhibitory in 1-2 week studies, anti-inflammatory agents did not eliminate the inflammation and it took 8 weeks of a constant subdural infusion of an effective agent to reduce numbers of phagocytic macrophages to the low levels observed in untreated rats at 16 weeks post-SCI.

The following parameters of a successful treatment to inhibit and eliminate the severe inflammation need to be considered in a neuroprotective therapy; (i) a potent anti-inflammatory agent that can reduce numbers of macrophages in the lesion; (ii) the route of administration effective in delivery of a candidate drug to the lesion. While intralesional administration has proven effective via subdural infusion and also by delivery of an anti-inflammatory agent from an implanted hydrogel it involves invasive neurosurgery. A systemic administration, such as oral or intravenous, while much less invasive and more practical to administer, needs to take into consideration the effect of inflammatory damage to the blood brain barrier (BBB) and to the brain spinal cord barrier (BSCB) and the damage-counteracting effect of astroglial reaction directed at restoring the BBB and/or BSCB. (iii) The duration of sustained administration of an anti-inflammatory agent has to be sufficient to eliminate macrophages from the lesion. A successful anti-inflammatory treatment in neurotrauma (SCI and TBI) and stroke will reduce the damage to the CNS around the initial traumatic, ischemic lesion and will accelerate elimination of edema leading to reduction in neurologic deficits and improved quality of life in acute patients, particularly those where the traumatic or ischemic injury is not extensive. In patients with large SCI, TBI or stroke lesions, an effective anti-inflammatory treatment may not restore enough neurological function by itself for acceptable quality of life, but it will allow for the application of tissue engineering therapies leading to neuroregeneration and greater degree of restoration of neurologic function. Such neuroengineering therapies are not considered possible when the severe destructive inflammation is active.

In view of the foregoing, it would be desirable to provide a method to reduce neuroinflammation associated with disease in mammals.

### SUMMARY OF THE INVENTION

The present invention generally relates to neuroinflammation and discloses a therapeutic method whereby administration of a protein improves pathologic outcomes in spinal cord injury (SCI), traumatic brain injury (TBI), stroke, and also in cerebral edema and spinal cord edema. Since the pathogenesis of neurodegenerative diseases including but not limited to Alzheimer's disease, frontotemporal dementia, Parkinson's disease, amyotrophic lateral sclerosis, immune mediated diseases including but not limited to multiple sclerosis and neuromyelitis optica and cerebrospinal infectious diseases including viral, bacterial, fungal, protozoal and parasitic involves neuroinflammation, systematic administration of the protein is expected to have an anti-inflammatory and neuroprotective therapeutic effect in these diseases as well.

Thus, a method to reduce neuroinflammation in mammals has now been developed. The method is useful to treat disease initiated, for example, by spinal cord injury (SCI), traumatic brain injury (TBI), stroke, infection and other causes of neuroinflammation, and optionally, to treat cerebral edema and spinal cord edema.

In one aspect, a method of treating or reducing neuroinflammation in a mammal is provided comprising administration to the mammal of non-immunogenic protein having anti-inflammatory activity.

In another aspect, a method of treating or reducing neuroinflammation in a mammal is provided comprising administration to the mammal of non-immunogenic protein having anti-inflammatory activity in conjunction with xanthohumol, a functionally equivalent variant or a pharmaceutically effective salt thereof.

These and other aspects of the invention will become apparent in the detailed description that follows by reference to the following figures.

### BRIEF DESCRIPTION OF DRAWINGS

FIG. 1. Shows the dose-response to treatment with rice protein or xanthohumol or both analysed in the Macrophage Counts in the Cavity of Injury (COI) in SCI rats at 7 days after the SCI. Lower counts of macrophages are interpreted as an anti-neuroinflammatory effect of a treatment. The values represent the average counts of macrophages with the control group of rats administered native, untreated jello cubes expressed as 100%. The top graph indicates a macrophage-lowering effect of rice protein in a dose-dependent fashion, ANOVA p<0.001. The middle graph indicates the dose-dependent macrophage-lowering effect of xanthohumol, ANOVA p<0.001. The lower graph indicates a dose-dependent effect of xanthohumol with 24 mg of rice protein in each dose on lowering counts of macrophages, ANOVA p<0.001.

FIG. 2. Shows micrographs of cross sections of the rat spinal cord at 7 days after the injury stained with luxol fast blue and counterstained with hematoxylin and eosin (LFB+H&E). The left column of micrographs (A, C, E) shows at low magnification the entire section of the spinal cord with the star indicating the cavity of injury (COI) and the arrowheads indicating the margin of the COI surrounded by the spinal cord. The right column of micrographs (B, D, F) shows at high magnification the margin (arrowheads) of the COI (star) with large cells containing a large round, oval, sometimes subcleaved nucleus surrounded by an abundant cytoplasm often encompassing granules of myelin and/or red blood cells. These phagocytic cells are interpreted as macrophages. While in the rats treated with native jello (A, B) high numbers of macrophages are packed in the COI, in rats treated with rice protein, 24 mg per jello cube (C, D) and with rice protein, 24 mg per jello cube in addition to 2 mg xanthohumol per jello cube (E, F), macrophages are more sparse and leave much of the debris un-phagocytized. The lower number of macrophages indicate an anti-neuroinflammatory effect of both types of treatments.

### DETAILED DESCRIPTION OF THE INVENTION

A method of reducing neuroinflammation in a mammal is provided comprising administration of non-immunogenic protein having anti-inflammatory activity.

The term “neuroinflammation” refers to inflammation of the tissue of the central nervous system (CNS) comprising the brain and spinal cord. Neuroinflammation may occur as a result of a number of causes, such as, microbial infection, traumatic brain injury, spinal cord injury, ageing, toxic metabolites, or autoimmunity, and results in recruitment of immune cells, such as macrophages, as well as inflammatory factors.

The present method of reducing neuroinflammation comprises administration of non-immunogenic protein having anti-inflammatory activity. The term “anti-inflammatory” with respect to the activity of the protein refers to its ability to inhibit, or at least reduce, inflammation in a mammal. Such proteins may be animal or plant-based. Preferred protein for use in the method also exhibit anti-oxidant properties. The amino acid profile of suitable non-immunogenic proteins comprises increased levels of sulfur-containing amino acids, for example, greater than 1 g/100 g, such as 2, 3, 4 or 5 g/100 g protein, of sulfur-containing amino acids including cysteine, methionine and mixtures thereof. The protein may also comprise reduced levels of amino acids such as lysine, threonine and/or tryptophan, such as less than 6, 5, 4 or 3 g/100 g of the protein. In embodiments, the protein is a plant-based protein such as rice protein, hemp protein, wheat protein, corn protein or potato protein.

In one embodiment, the protein is rice protein. Rice protein comprises glutelin (60-80%), globulin (5-13%), albumin (4-22%) and prolamin (1-5%). Rice protein is anti-inflammatory and at least its albumin component provides anti-oxidant activity. Rice protein comprises 2-4 g/100 g of sulfur-containing amino acids, i.e. methionine and cysteine. Rice protein is prepared by separating the protein component from the carbohydrate component to form a protein isolate. A number of methods may be utilized to obtain rice protein isolate including chemical extraction such as aqueous or alkali extraction methods, enzymatic extraction using carbohydrate—and starch-hydrolyzing enzymes. Physical extraction may also be utilized to obtain rice protein isolate including methods such as high-speed blending, sonication freeze-thaw and microfluidization techniques. Rice protein may be obtained from various types of rice including brown rice, red rice, gold rice, black rice or white rice.

The selected protein may be administered to a mammal either alone or in combination with at least one pharmaceutically acceptable adjuvant, for use in treatments in accordance with embodiments of the invention. The expression “pharmaceutically acceptable” means acceptable for use in the medical pharmaceutical and veterinary arts, i.e. not being unacceptably toxic or otherwise unsuitable. Examples of pharmaceutically acceptable adjuvants are those used conventionally with peptide-based drugs, such as diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents may be added to the composition to prevent microbial growth over prolonged storage periods.

A therapeutically effective amount of the protein is administered to a mammal to treat neuroinflammation. The term “treat”, “treating” or “treatment” as used herein with respect to neuroinflammation refers to the inhibition of neuroinflammation, or the reduction, amelioration or lessening of symptoms associated with neuroinflammation. As used herein, the term “mammal” is meant to encompass, without limitation, humans, and non-human mammals such as dogs, cats, horses, cattle, pigs, sheep, goats and the like. The term “therapeutically effective amount” is an amount of the protein sufficient to treat neuroinflammation as evidenced by at least a decrease in macrophage count (i.e. number of macrophages) at the site or cavity of injury by at least about 10%, 15%, 20%, 25%, 30%, 35%, 40% or more as compared to macrophage numbers at the site of injury in an untreated control, while not exceeding an amount which may cause significant adverse effects. In this regard, the number of macrophages is to be reduced in the site or cavity of injury to achieve neuroprotective and edema-reducing effects, while retaining sufficient (albeit reduced) macrophage numbers in order for phagocytosis and removal of myelin-rich debris to occur, which also aids in reducing neuroinflammation. Dosages of the protein that are therapeutically effective will vary with many factors including the nature of the condition to be treated as well as the particular individual being treated and the mode of administration. Appropriate oral dosages of the protein may be in the range of about 1-500 mg/kg body weight per day. For example, for small mammals such as rats, a dosage of about 1-200 mg/kg, such as 15-140 mg/kg body weight, 3 times per day, may be utilized, while dosages suitable for oral administration to humans may be in the range of 1-50 mg/kg, such as 3-20 mg/kg bodyweight 3 times a day.

In the present treatment, the protein may be administered by any route suitable to increase the plasma levels thereof and to target the site of injury. Examples of suitable administrable routes include, but are not limited to, oral, subcutaneous, intravenous, intraperitoneal, intranasal, enteral, topical, sublingual, intramuscular, intra-arterial, intramedullary, intrathecal, inhalation, ocular, transdermal, vaginal or rectal means. Depending on the route of administration, the protein may be delivered via a system that prevents undesirable degradation thereof by enzymes, acids or by other conditions that may affect the therapeutic activity thereof. A delivery system may also be selected to prevent exposure of the protein to conditions which may induce precipitation. The protein may, thus, be coated or encased in a protective material, or may be delivered by a vehicle such as a liposome, microsphere or nanostructure, or other suitable delivery system.

In another embodiment, the selected protein may be administered in conjunction with xanthohumol, a functionally equivalent variant thereof, or a pharmaceutically acceptable salt thereof. Thus, the protein may be administered in combination with xanthohumol, variant or salt thereof, together with or separately, simultaneously or at different times, using the same or different administrable routes. In an embodiment, the selected protein and xanthohumol are administered orally at the same time. In another embodiment, the xanthohumol is administered via a delivery vehicle, either together with or separately from, the selected protein, such as rice protein, to prevent the precipitation thereof on administration.

The term “functional equivalent variant” as it relates to xanthohumol includes naturally and non-naturally occurring variants thereof that retain the biological activity of native xanthohumol, also known as 2′,4,4′-trihydroxy-6′-methoxy-3′-(3-methylbut-2-en-1-yl) chalcone. The variant need not exhibit identical activity to native xanthohumol but will exhibit sufficient activity to render it useful to treat neuroinflammation, e.g. at least about 25% of the biological activity of native xanthohumol, and preferably at least about 50% or greater of the biological activity of native xanthohumol, to reduce macrophage number. Such functionally equivalent variants may result naturally or may result from non-naturally occurring synthetic alterations made to native xanthohumol to render functionally equivalent variants which may have more desirable characteristics for use in a therapeutic sense, for example, increased activity or stability as compared to native xanthohumol. Non-naturally occurring variants of native xanthohumol include analogues, fragments and derivatives thereof such as isoxanthohumol, hydrogenated derivatives, e.g. α,β-dihydroxanthohumol (DXN) and tetrahydroxanthohumol (TXN) and 8-prenylnaringenin.

Xanthohumol for use in the present method may be in the form of a pharmaceutically acceptable salt. A “pharmaceutically acceptable salt” refers to a salt that retains the desired biological activity of the parent compound and does not impart any undesired toxicological effects (see e.g., Berge, S. M. et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Xanthohumol for use in the present method may be obtained naturally, for example, from the Humulus lupulus plant. Alternatively, xanthohumol may be chemically synthesized using well-established chemical synthetic techniques.

Xanthohumol is used in combination with the selected protein, such as rice protein, in an amount that facilitates, and preferably enhances, the treatment of neuroinflammation. Dosages in the range of about 0.01 mg to 100 mg/kg bodyweight of xanthohumol may be used, for example 0.1 to 50 mg/kg bodyweight dosages daily. In embodiments, 1-20 mg/kg bodyweight 3 times daily is used in small mammals such as rats, while dosages 0.1-10 mg/kg bodyweight, such as 0.2-2 mg/kg, 3 times daily is used in humans.

When utilizing a combination of a selected anti-inflammatory protein such as rice protein together with xanthohumol, or derivative or salt thereof, in the present method, the dosage of xanthohumol utilized is substantially less than the dosage of the protein. For example, the ratio of xanthohumol to protein for use in the method may be in the range of about 1:5 to about 1:20 xanthohumol to protein, including ratios of 1:6, 1:7, 1:8, 1:9, 1:10, 1:11, 1:12, 1:13, 1:14, 1:15 or greater of xanthohumol to protein. The combination of the protein with xanthohumol advantageously permits reduced amounts of xanthohumol to be used in the present treatment.

In one embodiment, a dosage of xanthohumol in the range of 0.05-10 mg/kg bodyweight is utilized with a dosage of protein in the range of 0.5-150 mg/kg. In another embodiment, a dosage of xanthohumol in the range of 0.1-10 mg/kg bodyweight is utilized with a dosage of protein in the range of 1-110 mg/kg.

The present invention advantageously provides a method that has been determined to effectively treat neuroinflammation. The administration of a selected anti-inflammatory protein alone or together with xanthohumol crosses the blood-spinal cord barrier, and therefore, the blood brain barrier, to result in a reduction of the number of macrophages at the site of injury, thereby reducing inflammation. The method is effective to treat a number of disease conditions in which neuroinflammation plays a role, including but not limited to, traumatic brain injury, spinal cord injury, including brain and spinal cord injury sustained during surgical resection and in therapeutic cerebral irradiation, vascular disease such us in stroke or cerebral vascular aneurysm, neurodegenerative disease such as Alzheimer's disease, frontotemporal dementia, Parkinson's disease or amyotrophic lateral sclerosis, auto-immune disease of the central nervous system including but not limited to multiple sclerosis and neuromyelitis optica, and infectious disease resulting from viral, bacterial, fungal and parasitic myeloencephalitides. The present methods are also effective to inhibit vasogenic edema associated with neuroinflammation initiated by spinal cord injury, traumatic brain injury, surgical resection injury, therapeutic cerebral irradiation, stroke, cerebral vascular aneurysm, multiple sclerosis, neuromyelitis optica, and viral, bacterial, fungal and parasitic myeloencephalitides.

Embodiments of the invention are described in the following specific example which is not to be construed as limiting.

Example—Treatment of Neuroinflammation in an Animal Model of Spinal Cord Injury

A study was performed in a rat model of spinal cord injury (SCI) to determine the efficacy of a non-immunogenic protein isolate having anti-inflammatory and anti-oxidant activity alone or in combination with xanthohumol to treat neuroinflammation.

SCI model—Healthy male Long Evans rats aged 12 weeks, 340-390 g, were offered a fruit (strawberry or raspberry) flavored jello cube once a day, for 1 week prior to the surgery and were separated into individual cages 3 days before the surgery. Rats were induced with 5% isoflurane in 95% oxygen flowing at a rate of 1 liter per minute and maintained at 3.5% isoflurane in 96.5% oxygen. The anaesthetized rats had the skin on their backs shaved and prepared for surgery with 70% ethyl alcohol and 10% iodine swabs. The skin was cut over the caudal thoracic and lumbar spine and spinal muscles dissected from the vertebral spine of the thoracic 10 (T10) vertebrum and the dorsal arches of this vertebrum removed. A 3Fogarty catheter was inserted via this laminectomy over the intact dura towards the head to place the caudal edge of the 3 mm long balloon at 1 cm rostral to the laminectomy. The balloon was inflated with 15 μL of sterile saline for 3 minutes, then deflated and the catheter removed. The spinal muscles were closed with absorbable sutures over the laminectomy and the skin incision was closed with nylon non-absorbable sutures. Before awakening, the rats were administered; 50 μL of a painkiller Anafen (ketoprofen, 100 mg/mL, Merial) subcutaneously, and 50 μL of Baytril antibiotic (enrofloxacin 50 mg/mL, Bayer) intramuscular, and 3 mL of saline subcutaneous.

Treatments—The fruit jello cubes were prepared by diluting the jello powder, in post-boiling water and poured at 5 mL into plastic cube forms, then let to set in the refrigerator at 4° C.

Rice protein (CONVENTIONAL ORYZATEIN® SILK 90, AXIOM Foods, Los Angeles, CA, U.S.A.) was suspended in cold tap water and then added to liquid native jello to obtain a content of 6 mg, 12 mg, 24 mg or 48 mg of rice protein per 5 mL jello cube. The jello cubes were left to set in the refrigerator at 4° C.

Hop-derived xanthohumol (Hop-RXn, BioActive-Tech, Lublin Poland) was diluted in 100% ethyl alcohol and a volume added to 5 mL liquid native jello or to 5 mL jello with rice protein, 24 mg, to obtain 0.10 mg, 0.33 mg, 1.0 mg, 2.0 mg and 4.0 mg xanthohumol per jello cube. Liquid cubes were placed in the refrigerator at 4° C. to set.

Immediately after waking up from the anaesthesia post-SCI surgery, rats were offered a jello cube and then every 8 hours for 7 days. The ingestion of all jello cubes was noted to occur in all study rats after the surgery. Rats were divided into control, rice protein, and rice protein+xanthohumal treatment groups. Each treatment group had n=8 rats.

Given the invasive nature of the SCI model, an ethical endpoint was instituted. A rat with distended urinary bladder that was impossible to express or was ruptured, or with severe dehydration and with hypothermia and lethargy was humanely euthanized and not used in the study. The rats were administered Anafen painkiller once daily for 2 days post-surgery and rats with distended urinary bladder were given Baytril antibiotic once daily for 5 days post-surgery. Rats with moderate dehydration were administered 5-10 mL saline subcutaneously once or twice a day as needed. Rats with distended urinary bladder and micturition resulting in soiling of the perineal area had the bladder gently manually expressed once or twice a day and had a bath of the hind end in warm clean tap water done every day until normal bladder function returned and micturition stopped.

At 7 days post-surgery, the SCI rats were overdosed with the sodium pentobarbital (80 mg/kg b.w.) administered intraperitoneally. When in deep plane of anaesthesia, the chest was cut open, 100 international units of heparin sodium injected into the left heart ventricle and a cannula with flowing lactated Ringer's solution inserted into the left ventricle while the right auricle was cut open. After the blood was washed out, the flow of the lactated Ringer's solution was replaced by that of phosphate buffered formalin and the carcass was fixed. The spine was removed, postfixed in formalin overnight and then placed in formalin supplemented with 8% EDTA, pH 7.0, to decalcify the spinal vertebrae. The decalcifying solution was replaced with fresh solution every 2 days for 2 weeks. Once soft, the spine was cut perpendicular to its long axis into 3 mm thick segments starting from laminectomy rostrally to include the SCI lesion. Eight segments were processed in rising concentrations of ethyl alcohol and xylene, embedded in paraffin wax, cut 5 μm thick and mounted on the glass slide. The sections were stained with luxol fast blue and counterstained with hematoxylin and eosin (LFB+H&E) and cover-slipped. Stained sections were examined by an experienced experimental neuropathologist (the author) under a Nikon Eclipse 50i light microscope and the spinal cord photographed. At 40× magnification a margin of one COI per section, including 20% of the spinal cord and 80% of the COI, was digitally photographed at 40× magnification. The images were then analyzed. Macrophages, large cells with a round, oval, sometimes subcleaved nucleus with abundant cytoplasm containing granules of myelin debris and/or red blood cells, were counted. The counts were averaged for each rat and these averages were then averaged for each treatment group.

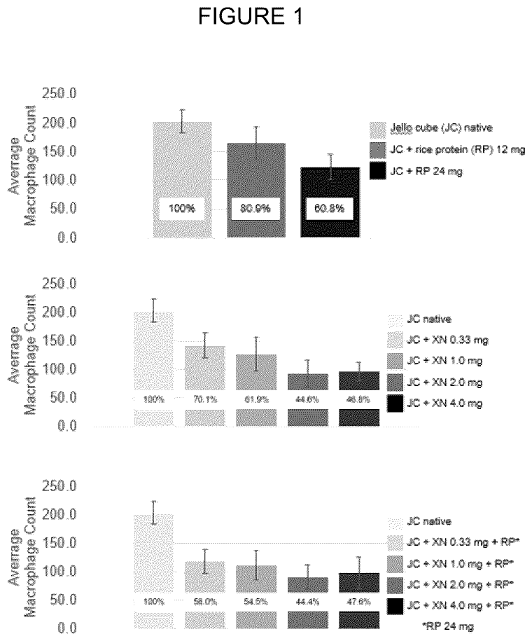

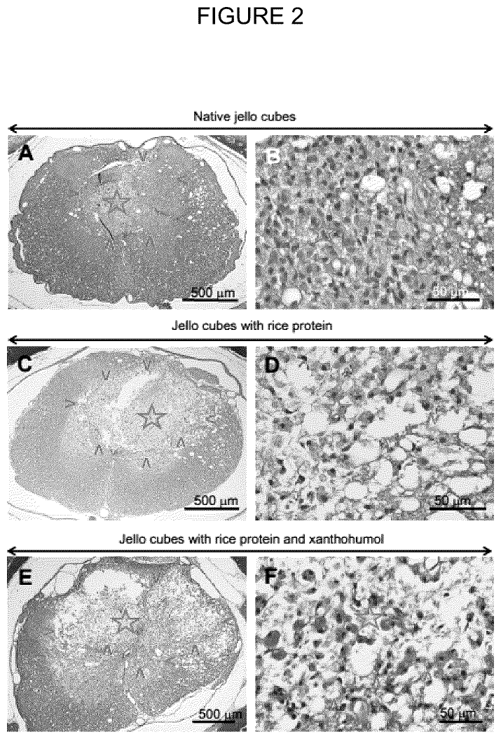

Results—Results of this study on the anti-inflammatory effect of rice protein and/or xanthohumol administered orally to rats with the SCI are demonstrated in FIGS. 1 and 2.

Rice protein had an anti-inflammatory effect in a dose-dependent manner. It was shown to lower the macrophage counts to 80% of the macrophage count in untreated control at 12 mg protein per cube and lowered to 60% of the control macrophage count at 24 mg protein per cube.

Xanthohumol had an anti-inflammatory effect in a dose-dependent fashion up to the 2 mg xanthohumol cubes which exhibited the maximum lowering of macrophages in the COI of 44% of the control macrophage count. In other words, the addition of increasing doses of xanthohumol to jello cubes containing 24 mg of rice protein resulted in an increased macrophage lowering effect reaching a maximum of 44% at 2 mg of xanthohumol per cube.

Discussion—Oral administration of rice protein with and without the addition of xanthohumol is described in the rat model of the SCI. An anti-inflammatory effect of rice protein in the rat SCI indicates it crossing the blood-spinal cord barrier and by implication, the blood brain barrier. This effect is dose-dependent for rice protein alone and in combination with xanthohumol as determined by the quantitative histologic test: standardized macrophage count in the cavity of injury (COI).

1. A method of treating or reducing neuroinflammation in a mammal comprising administration to the mammal of non-immunogenic protein having anti-inflammatory activity, wherein the protein comprises at least about 1 g/100 g protein of sulfur-containing amino acids selected from cysteine, methionine and a combination thereof.

2. The method of claim 1, wherein the protein is a plant-based protein selected from rice protein, hemp protein, wheat protein, corn protein or potato protein.

3. The method of claim 1, wherein the protein comprises 2-4 g/100 g protein of sulfur-containing amino acids.

4. The method of claim 1, wherein the protein has antioxidant activity.

5. The method of claim 1, wherein the protein is rice protein.

6. The method of claim 5, wherein the protein is administered orally.

7. The method of claim 1, wherein the neuroinflammation is reduced due to a reduction in the number of macrophages of a site of injury.

8. The method of claim 7, wherein the number of macrophages are reduced by at least about 10% in comparison to an untreated control.

9. The method of claim 1, wherein the protein is administered at a dosage in the range of about 1-500 mg/kg body weight per day.

10. The method of claim 1, to treat traumatic brain injury, spinal cord injury, vascular disease, neurodegenerative disease, auto-immune disease of the central nervous system, and infectious disease resulting from viral, bacterial, fungal or parasitic myeloencephalitides.

11. The method of claim 1, to treat vasogenic edema.

12. The method of claim 1, wherein the protein is administered in conjunction with xanthohumol, a functionally equivalent variant or a pharmaceutically effective salt thereof.

13. The method of claim 12, wherein the protein is administered together with xanthohumol.

14. The method of claim 12, wherein the protein is rice protein administered orally.

15. The method of claim 13, wherein the protein is rice protein and the protein and xanthohumol are administered orally.

16. The method of claim 13, wherein the protein is administered at a dosage in the range of about 1-500 mg/kg body weight per day and the xanthohumol is administered at a dosage in the range of about 0.01 mg to 100 mg/kg bodyweight.

17. The method of claim 12, wherein the ratio of xanthohumol to protein administered is about 1:20 xanthohumol to protein.

18. The method of claim 13, to treat traumatic brain injury, spinal cord injury, vascular disease, neurodegenerative disease, auto-immune disease of the central nervous system, and infectious disease resulting from viral, bacterial, fungal or parasitic myeloencephalitides.

19. The method of claim 13, to treat vasogenic edema.

20. Use of non-immunogenic protein having anti-inflammatory activity to treat neuroinflammation in a mammal, optionally in conjunction with xanthohumol, a functionally equivalent variant or a pharmaceutically effective salt thereof, wherein the protein comprises at least about 1 g/100 g protein of sulfur-containing amino acids selected from cysteine, methionine and a combination thereof, and the treatment results in a reduction of the number of macrophages at a site of injury.
